(19) United States
(12) Patent Application Publication
(10) Pub. No.: US 2024/0145778 A1
(43) Pub. Date: May 2, 2024

(54) RECHARGEABLE LITHIUM BATTERY

(71) Applicant: SAMSUNG SDI CO., LTD., Yongin-si (KR)

(72) Inventors: Yeji YANG, Yongin-si (KR); Hyunbong CHOI, Yongin-si (KR); Sundae KIM, Yongin-si (KR); Sangwoo PARK, Yongin-si (KR); Dahyun KIM, Yongin-si (KR); Hongryeol PARK, Yongin-si (KR); Sanghoon KIM, Yongin-si (KR)

(21) Appl. No.: 18/459,275

(22) Filed: Aug. 31, 2023

(30) Foreign Application Priority Data
Oct. 13, 2022 (KR) 10-2022-0131830

(51) Int. Cl. H01M 10/0567; C07C 49/14; C07C 49/167; H01M 4/36; H01M 4/38; H01M 4/505; H01M 4/525; H01M 4/587; H01M 10/0525; H01M 10/0569; H01M 10/42

(52) U.S. Cl. CPC H01M 10/0567; C07C 49/14; C07C 49/167; H01M 4/364; H01M 4/386; H01M 4/505; H01M 4/525; H01M 4/587; H01M 10/0525; H01M 10/0569; H01M 10/4235; H01M 2004/028; H01M 2300/0037

(57) ABSTRACT

Disclosed are a rechargeable lithium battery including an electrolyte solution including a non-aqueous organic solvent, a lithium salt, and an additive; a positive electrode including a positive electrode active material; and a negative electrode including a negative electrode active material, wherein the non-aqueous organic solvent includes ethylene carbonate in an amount of less than about 5 wt %, and the additive includes a compound represented by Chemical Formula 1, and the positive electrode active material is represented by Chemical Formula 2. Chemical Formula 1 and Chemical Formula 2 are the same as defined in the specification.

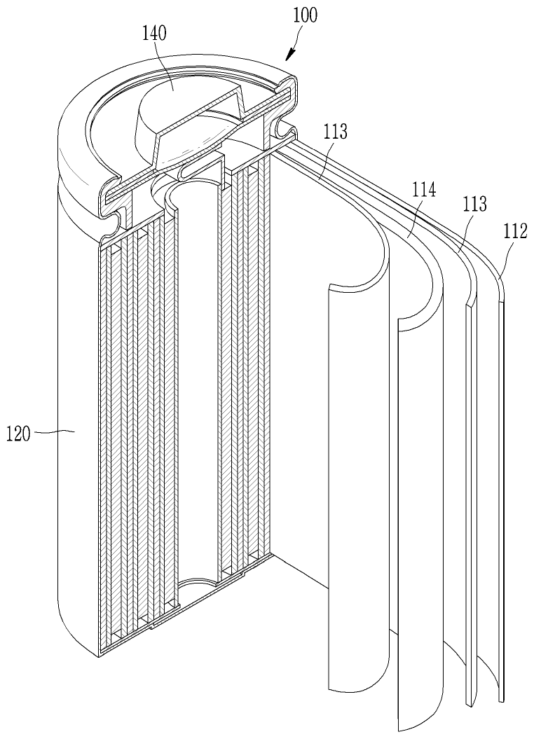

### CROSS-REFERENCE TO RELATED APPLICATION

This application claims priority to and the benefit of Korean Patent Application No. 10-2022-0131830 filed in the Korean Intellectual Property Office on Oct. 13, 2022, the entire content of which is hereby incorporated by reference.

### BACKGROUND

### 1. Field

Embodiments of this disclosure relate to a rechargeable lithium battery.

### 2. Description of the Related Art

A rechargeable lithium battery may be recharged and has three or more times as much energy density per unit weight as a conventional lead storage battery, nickel-cadmium battery, nickel hydrogen battery, nickel zinc battery and the like. It may be also charged at a high rate, and thus, is commercially manufactured for a laptop, a cell phone, an electric tool, an electric bike, and the like, and research on improvement of additional energy density have been actively made.

As IT devices (e.g., electronic devices) are increasingly achieving high-performance, high-capacity batteries are desired, but the high capacity may be realized through expansion of a voltage region, which may increase energy density, but in the high-voltage region, there is a problem of deteriorating performance of a positive electrode due to oxidization of an electrolyte solution.

For example, because a cobalt-free lithium nickel manganese-based oxide as a positive electrode active material includes not cobalt but nickel, manganese, and the like as a main component in a positive electrode active material composition, a positive electrode including this cobalt-free lithium nickel manganese-based oxide as a positive electrode active material is economical and realizes high energy density, and thus draws, a large amount of attention as a next generation positive electrode active material.

However, when the positive electrode including the cobalt-free lithium nickel manganese-based oxide is used in a high-voltage environment, transition metals are eluted therefrom due to structural collapse of the positive electrode, which may cause a problem such as gas generation inside a cell, capacity reduction, and the like. The transition metals tend to be more eluted in a high-temperature environment and precipitated on the surface of a negative electrode, and thus, cause a side reaction, which leads to an increase in battery resistance, deterioration of cycle-life and output characteristics of a battery.

Accordingly, when the positive electrode including the cobalt-free lithium nickel manganese-based oxide is used, an electrolyte solution applicable under high-voltage and high-temperature conditions is desired.

### SUMMARY

An embodiment provides a rechargeable lithium battery exhibiting improved high-voltage characteristics and high-temperature characteristics by combining a layered positive electrode active material including a cobalt-free lithium nickel manganese-based oxide and an electrolyte solution effectively protecting a positive electrode including the positive electrode active material to reduce transition metal elution under high-voltage and high-temperature conditions and suppress or reduce structural collapse of the positive electrode.

An embodiment provides a rechargeable lithium battery including an electrolyte solution including a non-aqueous organic solvent, a lithium salt, and an additive; a positive electrode including a positive electrode active material; and a negative electrode including a negative electrode active material,
wherein the non-aqueous organic solvent includes ethylene carbonate in an amount of less than about 5 wt %,
the additive includes a compound represented by Chemical Formula 1, and
the positive electrode active material is represented by Chemical Formula 2.

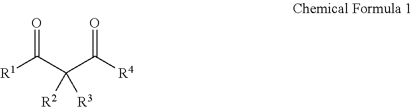

In Chemical Formula 1,
R¹ to R⁴ are each independently hydrogen, a halogen, a cyano group, a substituted or unsubstituted C1 to C20 alkyl group, a substituted or unsubstituted C6 to C20 aryl group, or a substituted or unsubstituted C2 to C30 heterocyclic group; Chemical Formula 2

LiₐNiₓMn_{1-x-y}A_{y}O_{2±b}X_{c}
wherein, in Chemical Formula 2,
0.9≤a≤1.2, 0≤b≤0.1, 0≤c≤0.1, 0.5≤x≤0.95, and 0≤y<0.3,
A is at least one element selected from B, Na, Mg, Al, Ti, and Si, and
X is at least one element selected from S, F, P, and Cl.

The non-aqueous organic solvent may be composed of only chain carbonate.

The chain carbonate may be represented by Chemical Formula 3.

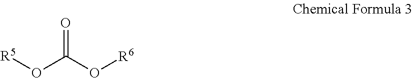

In Chemical Formula 3
R⁵ and R⁶ are each independently a substituted or unsubstituted C1 to C20 alkyl group.

The non-aqueous organic solvent may be a mixed solvent comprising at least two of dimethyl carbonate (DMC), diethyl carbonate (DEC), dipropyl carbonate (DPC), methylpropyl carbonate (MPC), ethylpropyl carbonate (EPC), and ethylmethyl carbonate (EMC).

The non-aqueous organic solvent may include ethylmethyl carbonate (EMC) and dimethyl carbonate (DMC) in a weight ratio of about 0:100 to about 50:50.

At least one selected from R¹ to R⁴ in Chemical Formula 1 may be an unsubstituted C1 to C20 alkyl group, a C1 to C20 alkyl group substituted with an electron withdrawing group, an unsubstituted C6 to C20 aryl group, a C6 to C20 aryl group substituted with an electron withdrawing group, an unsubstituted C2 to C30 heterocyclic group, or a C2 to C30 heterocyclic group substituted with an electron withdrawing group.

The electron withdrawing group may be at least one selected from a halogen, an isocyanate group (—NCO), an isothiocyanate group (—NCS), a cyanate group (—OCN), a thiocyanate group (—SCN), a cyano group (—CN), an isocyano group (—NC), a —N═C═N— group, a —N═S═N— group, a nitro group (NO₂), a trifluoromethane (CF₃) group, a pentafluoroethane (C₂F₅) group, a trifluoromethane sulfonyl (SO₂CF₃) group, a pentafluoroethane sulfonyl (SO₂C₂F₅) group, a trifluoromethane sulfonate (SO₃CF₃) group, a pentafluoroethane sulfonate (SO₃C₂F₅) group, a pentafluorophenyl (C₆F₅) group, an acetyl (COCH₃) group, an ethyl ketone (COC₂H₅) group, a propyl ketone (COC₃H₇) group, a butyl ketone (COC₄H₉) group, a pentyl ketone (COC₅H₁₁) group, a hexyl ketone (COC₆H₁₃) group, an ethanoate (CO₂CH₃) group, a propanoate (CO₂C₂H₅) group, a butaneoate (CO₂C₃H₇) group, a pentanoate (CO₂C₄H₉) group, and a hexanoate (CO₂C₅H₁₁) group.

At least one selected from R² and R³ in Chemical Formula 1 may be hydrogen.

R² and R³ of Chemical Formula 1 may each be hydrogen.

The compound represented by Chemical Formula 1 may be selected from compounds of Group 1.

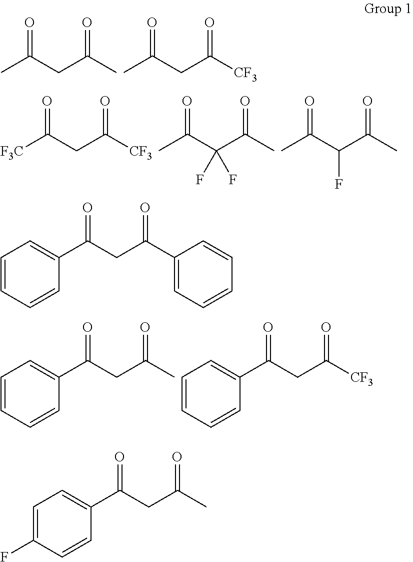

The compound represented by Chemical Formula 1 may be included in an amount of about 0.01 to about 5.0 parts by weight based on 100 parts by weight of the electrolyte solution for a rechargeable lithium battery.

The electrolyte solution may further include at least one other additives selected from vinylene carbonate (VC), fluoroethylene carbonate (FEC), difluoroethylene carbonate, chloroethylene carbonate, dichloroethylene carbonate, bromoethylene carbonate, dibromoethylene carbonate, nitroethylene carbonate, cyanoethylene Carbonate, vinylethylene carbonate (VEC), adiponitrile (AN), succinonitrile (SN), 1,3,6-hexane tricyanide (HTCN), propensultone (PST), propanesultone (PS), lithium tetrafluoroborate (LiBF₄), lithium difluorophosphate (LiPO₂F₂), and 2-fluorobiphenyl (2-FBP).

In Chemical Formula 2, x may be 0.8≤x≤0.95.

The positive electrode active material may include LiNi_{0.75}Mn_{0.25}O₂, LiNi_{0.80}Mn_{0.20}O₂, LiNi_{0.85}Mn_{0.15}O₂, LiNi_{0.90}Mn_{0.10}O₂ or LiNi_{0.95}Mn_{0.05}O₂, and a solid solution thereof.

The negative electrode active material may include at least one selected from graphite and a Si composite.

The rechargeable lithium battery may have a charging upper limit voltage of greater than or equal to about 4.35 V.

An embodiment uses a combination of a layered positive electrode active material including a cobalt-free lithium nickel manganese-based oxide and an electrolyte solution effectively protecting a positive electrode including the layered positive electrode active material to secure phase transition safety of the positive electrode even in a high-temperature and high-voltage environment and to reduce gas generation by suppressing or reducing decomposition of the electrolyte solution and a side reaction with electrodes and concurrently (e.g., simultaneously), to improve battery stability and cycle-life characteristics of a rechargeable lithium battery by suppressing or reducing an increase in internal battery resistance.

### BRIEF DESCRIPTION OF THE DRAWING

The accompanying drawing, together with the specification, illustrate embodiments of the subject matter of the present disclosure, and, together with the description, serve to explain principles of embodiments of the subject matter of the present disclosure.

The accompanying drawing is a schematic view showing a rechargeable lithium battery according to an embodiment.

### DETAILED DESCRIPTION

Hereinafter, a rechargeable lithium battery according to embodiments of the present disclosure will be described in more detail with reference to the accompanying drawing. However, these embodiments are examples, the present disclosure is not limited thereto and the scope of the present disclosure is defined by the scope of the appended claims, and equivalents thereof.

As used herein, when a definition is not otherwise provided “substituted” refers to replacement of at least one hydrogen of a substituent or a compound by deuterium, a halogen, a hydroxyl group, an amino group, a C1 to C30 amine group, a nitro group, a C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a C1 to C10 fluoroalkyl group, an isocyanate group (—NCO), an isothiocyanate group (—NCS), a cyanate group (—OCN), a thiocyanate group (—SCN), a cyano group (—CN), an isocyano group (—NC) or a combination thereof.

In some examples of the present disclosure, “substituted” refers to replacement of at least one hydrogen of a substituent or a compound by deuterium, a halogen, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C10 fluoroalkyl group, a C1 to C10 trifluoroalkyl group, or a cyano group. In addition, in some examples of the present disclosure, “substituted” refers to replacement of at least one hydrogen of a substituent or a compound by deuterium, a halogen, a C1 to C20 alkyl group, a C6 to C30 aryl group, a C1 to C10 fluoroalkyl group, a C1 to C10 trifluoroalkyl group, or a cyano group. In addition, in some examples of the present disclosure, “substituted” refers to replacement of at least one hydrogen of a substituent or a compound by deuterium, a halogen, a C1 to C5 alkyl group, a C6 to C18 aryl group, a C1 to C5 fluoroalkyl group, a C1 to C5 trifluoroalkyl group, or a cyano group. In addition, in some examples of the present disclosure, “substituted” refers to replacement of at least one hydrogen of a substituent or a compound by deuterium, a cyano group, a halogen, a methyl group, an ethyl group, a propyl group, a butyl group, a phenyl group, a biphenyl group, a terphenyl group, trifluoromethyl group, or a naphthyl group.

A rechargeable lithium battery may be classified as a lithium ion battery, a lithium ion polymer battery, or a lithium polymer battery depending on kinds of a separator and an electrolyte solution utilized. It also may be classified as a cylindrical, a prismatic, a coin-type, a pouch-type, or the like depending on a shape of the rechargeable lithium battery. In addition, it may be classified as a bulk type or a thin film type depending on a size of the rechargeable lithium battery. Structures and manufacturing methods for lithium ion batteries pertaining to this disclosure should be readily recognizable to those of ordinary skill in the art upon reviewing the present disclosure.

Herein, as an example of a rechargeable lithium battery, a cylindrical rechargeable lithium battery is described, but the present disclosure is not limited thereto. The accompany drawing schematically shows the structure of a rechargeable lithium battery according to an embodiment. Referring to the accompanying drawing, a rechargeable lithium battery 100 according to an embodiment includes a battery cell including a positive electrode 114, a negative electrode 112 facing the positive electrode 114, and a separator 113 between the positive electrode 114 and the negative electrode 112, an electrolyte solution impregnating the positive electrode 114, the negative electrode 112 and the separator 113, a battery case 120 housing the battery cell, and a sealing member 140 for sealing the battery case 120.

Hereinafter, a more detailed configuration of the rechargeable lithium battery 100 according to an embodiment is described.

The rechargeable lithium battery according to an embodiment of the present disclosure includes an electrolyte solution, a positive electrode, and a negative electrode.

The electrolyte solution may include a non-aqueous organic solvent, a lithium salt, and an additive, wherein the non-aqueous organic solvent includes ethylene carbonate in an amount of less than about 5 wt % (e.g., based on 100 wt % of the electrolyte solution), and the additive may include a compound represented by Chemical Formula 1.

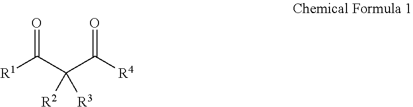

In Chemical Formula 1,
R¹ to R⁴ are each independently hydrogen, a halogen, a cyano group, a substituted or unsubstituted C1 to C20 alkyl group, a substituted or unsubstituted C6 to C20 aryl group, or a substituted or unsubstituted C2 to C30 heterocyclic group.

The positive electrode may include a positive electrode active material including a lithium nickel manganese-based oxide represented by Chemical Formula 2.

The lithium nickel manganese-based oxide represented by Chemical Formula 2 is a cobalt-free lithium nickel-manganese-based oxide and may be a layered positive electrode active material. As used herein, the term “cobalt-free lithium nickel-manganese-based oxide” may mean that the cobalt-free lithium nickel-manganese-based oxide is completely free of cobalt. In the case of a positive electrode active material including a cobalt-free lithium nickel manganese-based oxide, solvent decomposition and elution of transition metals, for example, Ni, may occur due to strong structural instability under a high-voltage condition.

Due to such an elution phenomenon of transition metals, deterioration and short-circuiting of the rechargeable lithium battery may occur, resulting in a decrease in cycle-life capacity of the rechargeable lithium battery and a rapid increase in resistance (e.g., electrical resistance).

However, in the case of using the aforementioned electrolyte solution together, it is possible to alleviate the decrease in the cycle-life capacity of the battery and the rapid increase in resistance.

In some embodiments, by using the aforementioned positive electrode active material in an electrolyte solution containing less than about 5 wt % of ethylene carbonate, specifically greater or equal to 0 wt % and less less than about 5 wt % of ethylene carbonate, the elution of transition metals may be effectively reduced under high voltage and high-temperature conditions, thereby suppressing or reducing collapse of the positive electrode structure and improving high-voltage characteristics and high-temperature characteristics of the rechargeable lithium battery.

The non-aqueous organic solvent serves as a medium for transmitting ions taking part in the electrochemical reaction of a battery.

The non-aqueous organic solvent may include a carbonate-based, ester-based, ether-based, ketone-based, alcohol-based, and/or aprotic solvent.

The carbonate-based solvent may include dimethyl carbonate (DMC), diethyl carbonate (DEC), dipropyl carbonate (DPC), methylpropyl carbonate (MPC), ethylpropyl carbonate (EPC), ethylmethyl carbonate (EMC), ethylene carbonate (EC), propylene carbonate (PC), butylene carbonate (BC), and/or the like. The ester-based solvent may include methyl acetate, ethyl acetate, n-propyl acetate, t-butyl acetate, methyl propionate, ethyl propionate, propyl propionate, decanolide, mevalonolactone, caprolactone, and/or the like. The ether-based solvent may include dibutyl ether, tetraglyme, diglyme, dimethoxyethane, 2-methyltetrahydrofuran, tetrahydrofuran, and/or the like. In addition, the ketone-based solvent may include cyclohexanone, 20 and/or the like. In addition, the alcohol-based solvent may include ethanol, isopropyl alcohol, and/or the like and the aprotic solvent may include nitriles such as R—CN (wherein R is a C2 to C20 linear, branched, or cyclic hydrocarbon group and includes a double bond, an aromatic ring or an ether bond), and/or the like, amides such as dimethyl formamide, dioxolanes such as 1,3-dioxolane, sulfolanes, and/or the like.

The non-aqueous organic solvents may be used alone or in combination with one or more of them, and when used in combination with one or more, the mixing ratio may be suitably or appropriately adjusted according to suitable or desired battery performance, which should be readily recognizable by those skilled in the art upon reviewing this disclosure.

For example, the non-aqueous organic solvent may include less than 5 wt % of ethylene carbonate (e.g., based on 100 wt % of the non-aqueous organic solvent or the carbonate-based solvent).

When the content of ethylene carbonate is greater than or equal to about 5 wt %, activity of Ni increases during high-voltage driving, the oxidation number of Ni tends to be reduced from tetravalent to divalent, and ethylene carbonate with low oxidation stability is oxidatively decomposed, resulting in Ni being eluted and deposited on the negative electrode.

As an example, the non-aqueous organic solvent may be composed of only chain carbonate (e.g., the non-aqueous organic solvent or the carbonate-based solvent may be substantially free or completely free of cyclic carbonates). In this case, as the resistance increase rate during high-temperature storage is significantly alleviated, excellent high-temperature storage characteristics may be implemented.

In this specification, the meaning of composed of only chain carbonates means including organic solvents belonging to the category of chain carbonates alone or in combination without being mixed together with cyclic carbonates and/or the like. For example, the non-aqueous organic solvent or the carbonate-based solvent may be substantially free of cyclic carbonates and the like such that cyclic carbonates are present in the non-aqueous organic solvent or the carbonate-based solvent, if at all, only as an incidental impurity. In some embodiments, the non-aqueous solvent is completely free of cyclic carbonates.

In an embodiment, the chain carbonate may be represented by Chemical Formula 3.

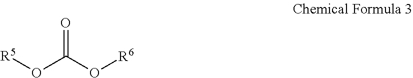

In Chemical Formula 3, R⁵ and R⁶ are each independently a substituted or unsubstituted C1 to C20 alkyl group.

For example, R⁵ and R⁶ in Chemical Formula 3 may each independently be a substituted or unsubstituted C1 to C10 alkyl group, and for example, R⁵ and R⁶ may each independently be a substituted or unsubstituted C1 to C5 alkyl group.

In an embodiment, R⁵ and R⁶ in Chemical Formula 3 may each independently be a substituted or unsubstituted methyl group, a substituted or unsubstituted ethyl group, a substituted or unsubstituted n-propyl group, a substituted or unsubstituted n-butyl group, a substituted or unsubstituted n-pentyl group, a substituted or unsubstituted iso-butyl group, or a substituted or unsubstituted neo-pentyl group.

For example, the non-aqueous organic solvent according to some embodiments may include at least two selected from dimethyl carbonate (DMC), diethyl carbonate (DEC), dipropyl carbonate (DPC), methylpropyl carbonate (MPC), ethylpropyl carbonate (EPC), and ethylmethyl carbonate (EMC).

The non-aqueous organic solvent according to some embodiments may be a mixed solvent of dimethyl carbonate (DMC) and ethylmethyl carbonate (EMC).

The non-aqueous organic solvent may include ethylmethyl carbonate (EMC) and dimethyl carbonate (DMC) in a weight ratio of about 0:100 to about 50:50.

Battery characteristics of the rechargeable lithium battery may be improved by including dimethyl carbonate (DMC) in the non-aqueous organic solvent in an amount of more than about 50 wt % (e.g., based on 100 wt % of the non-aqueous organic solvent or the carbonate-based solvent).

For example, the non-aqueous organic solvent may include ethylmethyl carbonate (EMC) and dimethyl carbonate (DMC) in a weight ratio of about 0:100 to about 40:60, about 0:100 to about 30:70, about 10:90 to about 40:60, or about 10:90 to about 30:70.

The non-aqueous organic solvent may further include an aromatic hydrocarbon-based solvent in addition to the carbonate-based solvent. In this case, the carbonate-based solvent and the aromatic hydrocarbon-based solvent may be mixed together in a volume ratio of about 1:1 to about 30:1.

The aromatic hydrocarbon-based solvent may include an aromatic hydrocarbon-based compound represented by Chemical Formula 4.

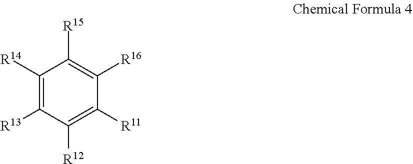

In Chemical Formula 4, R¹¹ to R¹⁶ are the same as or different from each other and are selected from hydrogen, a halogen, a C1 to C10 alkyl group, a C1 to C10 haloalkyl group, and a combination thereof.

Examples of the aromatic hydrocarbon-based solvent may include benzene, fluorobenzene, 1,2-difluorobenzene, 1,3-difluorobenzene, 1,4-difluorobenzene, 1,2,3-trifluorobenzene, 1,2,4-trifluorobenzene, chlorobenzene, 1,2-dichlorobenzene, 1,3-dichlorobenzene, 1,4-dichlorobenzene, 1,2,3-trichlorobenzene, 1,2,4-trichlorobenzene, iodobenzene, 1,2-diiodobenzene, 1,3-diiodobenzene, 1,4-diiodobenzene, 1,2,3-triiodobenzene, 1,2,4-triiodobenzene, toluene, fluorotoluene, 2,3-difluorotoluene, 2,4-difluorotoluene, 2,5-difluorotoluene, 2,3,4-trifluorotoluene, 2,3,5-trifluorotoluene, chlorotoluene, 2,3-dichlorotoluene, 2,4-dichlorotoluene, 2,5-dichlorotoluene, 2,3,4-trichlorotoluene, 2,3,5-trichlorotoluene, iodotoluene, 2,3-diiodotoluene, 2,4-diiodotoluene, 2,5-diiodotoluene, 2,3,4-triiodotoluene, 2,3,5-triiodotoluene, xylene, and a combination thereof.

The lithium salt dissolves in a non-aqueous organic solvent and acts as a source of lithium ions in the battery to enable basic operation of the rechargeable lithium battery and promotes movement of lithium ions between the positive electrode and the negative electrode. Representative examples of such lithium salts may include LiPF₆, LiBF₄, lithium difluoro(oxalate)borate (LiDFOB), LiPO₂F₂, LiSbF₆, LiAsF₆, Li(FSO₂)₂N (lithium bis(fluorosulfonyl)imide: LiFSI), LiC₄F₉SO₃, LiClO₄, LiAlO₂, LiAlCl₄, LiN(CₓF₂ₓ₊₁SO₂)(C_{y}F_{2y+1}SO₂), where x and y are integers selected from 1 to 20, LiCl, LiI, or LiB(C₂O₄)₂ (lithium bis(oxalato) borate: LiBOB).

A concentration of the lithium salt may be used within the range of about 0.1 M to about 2.0 M. When the lithium salt is included at the above concentration range, an electrolyte may have excellent performance and lithium ion mobility due to optimal electrolyte conductivity and viscosity.

At least one selected from R¹ to R⁴ in Chemical Formula 1 may be a C1 to C20 alkyl group optionally substituted with an electron withdrawing group, a C6 to C20 aryl group optionally substituted with an electron withdrawing group, or a C2 to C30 heterocyclic group optionally substituted with an electron withdrawing group.

The electron withdrawing group may be at least one selected from a halogen, an isocyanate group (—NCO), an isothiocyanate group (—NCS), a cyanate group (—OCN), a thiocyanate group (—SCN), a cyano group (—CN), an isocyano group (—NC), a —N═C═N— group, a —N═S═N— group, a nitro group (NO₂), a trifluoromethane (CF₃) group, a pentafluoroethane (C₂F₅) group, a trifluoromethane sulfonyl (SO₂CF₃) group, a pentafluoroethane sulfonyl (SO₂C₂F₅) group, a trifluoromethane sulfonate (SO₃CF₃) group, a pentafluoroethane sulfonate (SO₃C₂F₅) group, a pentafluorophenyl (C₆F₅) group, an acetyl (COCH₃) group, an ethyl ketone (COC₂H₅) group, a propyl ketone (COC₃H₇) group, a butyl ketone (COC₄H₉) group, a pentyl ketone (COC₅H₁₁) group, a hexyl ketone (COC₆H₁₃) group, an ethanoate (CO₂CH₃) group, a propanoate (CO₂C₂H₅) group, a butaneoate (CO₂C₃H₇) group, a pentanoate (CO₂C₄H₉) group, and a hexanoate (CO₂C₅H₁₁) group.

At least one selected from R² and R³ in Chemical Formula 1 may be hydrogen.

R² and R³ in Chemical Formula 1 may each be hydrogen.

The compound represented by Chemical Formula 1 may be selected from the compounds of Group 1.

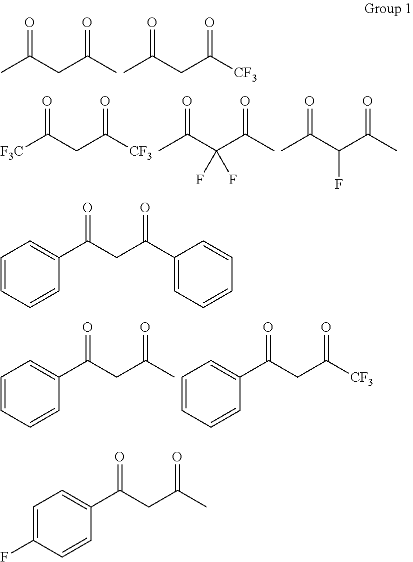

The additive may be included in an amount of about 0.01 to about 5.0 parts by weight based on 100 parts by weight of the electrolyte solution for a rechargeable lithium battery.

For example, the additive may be included in an amount of about 0.01 to about 3.0 parts by weight based on 100 parts by weight of the electrolyte solution for a rechargeable lithium battery.

For example, the additive may be included in an amount of about 0.1 to about 3.0 parts by weight, about 0.3 to about 3.0 parts by weight, about 0.5 to about 3.0 parts by weight, or about 0.5 to about 2.0 parts by weight based on 100 parts by weight of the electrolyte solution for a rechargeable lithium battery.

When the content range of the additive is as described above, it is possible to implement a rechargeable lithium battery having improved cycle-life characteristics and output characteristics by preventing or reducing an increase in resistance at high temperatures.

On the other hand, the electrolyte solution may further include at least one other additive selected from vinylene carbonate (VC), fluoroethylene carbonate (FEC), difluoroethylene carbonate, chloroethylene carbonate, dichloroethylene carbonate, bromoethylene carbonate, dibromoethylene carbonate, nitroethylene carbonate, cyanoethylene carbonate, vinylethylene carbonate (VEC), adiponitrile (AN), succinonitrile (SN), 1,3,6-hexane tricyanide (HTCN), propensultone (PST), propanesultone (PS), lithium tetrafluoroborate (LiBF₄), lithium difluorophosphate (LiPO₂F₂), and 2-fluorobiphenyl (2-FBP).

By further including the aforementioned other additives, the cycle-life may be further improved and/or gases generated from the positive electrode and the negative electrode may be suitably or effectively controlled during high-temperature storage.

The other additives may be included in an amount of about 0.2 to about 20 parts by weight, about 0.2 to about 15 parts by weight, or, for example, about 0.2 to about 10 parts by weight, based on 100 parts by weight of the electrolyte solution for a rechargeable lithium battery.

When the amount of other additives is as described above, the increase in film resistance may be minimized or reduced, thereby contributing to the improvement of battery performance.

The positive electrode includes a positive electrode current collector and a positive electrode active material layer formed on the positive electrode current collector, and the positive electrode active material layer includes a positive electrode active material.

The positive electrode active material may include cobalt-free lithium nickel manganese-based oxide represented by Chemical Formula 2.

LiₐNiₓMn_{1-x-y}A_{y}O_{2±b}X_{c} Chemical Formula 2

In Chemical Formula 2,
0.9≤a≤1.2, 0≤b≤0.1, 0≤c≤0.1, 0.5≤x≤0.95, and 0≤y<0.3,
A is at least one element selected from B, Na, Mg, Al, Ti, and Si, and
X is at least one element selected from S, F, P, and Cl.

In some embodiments, one or more having a coating layer on the surface of the lithium composite oxide may be used, or a mixture of the lithium composite oxide and a compound having a coating layer may be used. The coating layer may include at least one coating element compound selected from an oxide of a coating element, a hydroxide of the coating element, an oxyhydroxide of the coating element, an oxycarbonate of the coating element, and a hydroxy carbonate of the coating element. The compound for the coating layer may be amorphous or crystalline. The coating element included in the coating layer may include Mg, Al, K, Na, Ca, Si, Ti, V, Sn, Ge, Ga, B, As, Zr, or a mixture thereof. The coating layer may be provided by utilizing a method having no (or substantially no) adverse influence on properties of a positive electrode active material by using these elements in the compound. For example, the method may include any suitable coating method (e.g., spray coating, dipping, etc.), but is not described in more detail here because it should be readily recognizable to those of ordinary skill in the art upon reviewing this disclosure.

For example, x in Chemical Formula 2 may be 0.8≤x≤0.95.

For example, the positive electrode active material may include LiNi_{0.75}Mn_{0.25}O₂, LiNi_{0.80}Mn_{0.20}O₂, LiNi_{0.85}Mn_{0.15}O₂, LiNi_{0.90}Mn_{0.10}O₂, and/or LiNi_{0.95}Mn_{0.05}O₂, and/or a solid solution thereof.

The positive electrode active material may be included in an amount of about 90 wt % to about 98 wt % based on a total weight (100 wt %) of the positive electrode active material layer.

In an embodiment, the positive electrode active material layer may further include a binder and/or a conductive material (e.g., an electrically conductive material). Herein, each content of the binder and conductive material may be about 1 wt % to about 5 wt % based on a total weight (100 wt %) of the positive electrode active material layer.

The binder improves binding properties of positive electrode active material particles with one another and with a positive electrode current collector. Examples thereof may include polyvinyl alcohol, carboxylmethyl cellulose, hydroxypropyl cellulose, diacetyl cellulose, polyvinylchloride, carboxylated polyvinylchloride, polyvinylfluoride, an ethylene oxide-containing polymer, polyvinylpyrrolidone, polyurethane, polytetrafluoroethylene, polyvinylidene fluoride, polyethylene, polypropylene, a styrene-butadiene rubber, an acrylated styrene-butadiene rubber, an epoxy resin, nylon, and the like, but the binder is not limited thereto.

The positive electrode current collector may be Al, but is not limited thereto.

The negative electrode includes a negative electrode current collector and a negative electrode active material layer including the negative electrode active material formed on the negative electrode current collector.

The negative electrode active material includes a material that reversibly intercalates/deintercalates lithium ions, a lithium metal, a lithium metal alloy, a material capable of doping/dedoping lithium, and/or a transition metal oxide.

The material that reversibly intercalates/deintercalates lithium ions may include carbon material. The carbon material may be any suitable carbon-based negative electrode active material generally used in a rechargeable lithium battery. Examples of the carbon material include crystalline carbon, amorphous carbon, and a combination thereof. The crystalline carbon may be non-shaped, and/or sheet, flake, spherical, and/or fiber shaped natural graphite and/or artificial graphite. The amorphous carbon may be a soft carbon, a hard carbon, a mesophase pitch carbonized product, calcined coke, and/or the like.

The lithium metal alloy be an alloy of lithium and a metal selected from Na, K, Rb, Cs, Fr, Be, Mg, Ca, Sr, Si, Sb, Pb, In, Zn, Ba, Ra, Ge, Al, and Sn.

The material capable of doping and dedoping lithium may include Si, SiOₓ (0<x<2), a Si-Q alloy (wherein Q is selected from an alkali metal, an alkaline-earth metal, a Group 13 element, a Group 14 element excluding Si, a Group 15 element, a Group 16 element, a transition metal, a rare earth element, and a combination thereof), Sn, SnO₂, Sn—R¹¹ (wherein R¹¹ is an alkali metal, an alkaline-earth metal, a Group 13 element, a Group 14 element excluding Sn, a Group 15 element, a Group 16 element, a transition element, a rare earth element, or a combination thereof), and/or the like. At least one of them may be mixed together with SiO₂.

The elements Q and R¹¹ may be selected from Mg, Ca, Sr, Ba, Ra, Sc, Y, Ti, Zr, Hf, Rf, V, Nb, Ta, Db, Cr, Mo, W, Sg, Tc, Re, Bh, Fe, Pb, Ru, Os, Hs, Rh, Ir, Pd, Pt, Cu, Ag, Au, Zn, Cd, B, Al, Ga, Sn, In, TI, Ge, P, As, Sb, Bi, S, Se, Te, Po, and a combination thereof.

The transition metal oxide may be a vanadium oxide, a lithium vanadium oxide, and/or the like.

In some embodiments, the negative electrode active material may include at least one selected from graphite and a Si composite.

The Si composite may include a core including Si-based particles and an amorphous carbon coating layer, and the Si-based particles may include at least one selected from silicon particles, a Si—C composite, SiOₓ (0<x≤2), and a Si alloy.

For example, a void may be included in the center portion of the core including the Si-based particles, the radius of the center portion may correspond to about 30% to about 50% of the radius of Si composite, an average particle diameter of the Si composite may be about 5 μm to about 20 μm, and an average particle diameter of the Si-based particles may be about 10 nm to about 200 nm.

In the present specification, an average particle diameter(D50) may be a particle size at a volume ratio of 50% in a cumulative size-distribution curve.

When the average particle diameter of the Si-based particles is within the above range, volume expansion occurring during charging and discharging may be suppressed or reduced, and disconnection of a conductive path (e.g., a lithium conductive path) due to particle crushing during charging and discharging may be prevented or reduced.

The core including the Si-based particles may further include amorphous carbon, and at this time, the center portion does not include amorphous carbon, and the amorphous carbon may exist only in the surface portion of the Si composite (e.g., the center portion of the Si composite may be free of amorphous carbon).

At this time, the surface portion means a region from the outermost surface of the central portion (the center) to the outermost surface of the Si composite.

In addition, the Si-based particles are substantially uniformly included throughout the Si composite, and may be present at a substantially uniform concentration in the center and surface portions.

The amorphous carbon may be soft carbon, hard carbon, a mesophase pitch carbonized product, calcined coke, or a combination thereof.

For example, the Si—C composite may include silicon particles and crystalline carbon.

The silicon particles may be included in an amount of about 1 wt % to about 60 wt %, for example, about 3 wt % to about 60 wt % based on the total weight of the Si—C composite.

The crystalline carbon may be, for example, graphite, and, in some embodiments, may be natural graphite, artificial graphite, or a combination thereof.

An average particle diameter of the crystalline carbon may be about 5 μm to about 30 μm.

When the negative electrode active material includes graphite and Si composite together, the graphite and Si composite may be included as a mixture, and in this case, the graphite and Si composite may be included in a weight ratio of about 99:1 to about 50:50.

In some embodiments, the graphite and Si composite may be included in a weight ratio of about 97:3 to about 80:20, or about 95:5 to about 80:20.

The amorphous carbon precursor may be coal-based pitch, mesophase pitch, petroleum-based pitch, coal-based oil, petroleum-based heavy oil, and/or a polymer resin such as a phenol resin, a furan resin, and/or a polyimide resin.

In the negative electrode active material layer, the negative electrode active material may be included in an amount of about 95 wt % to about 99 wt % based on the total weight of the negative electrode active material layer.

In an embodiment, the negative electrode active material layer may include a binder, and optionally a conductive material (e.g., an electrically conductive material). In the negative electrode active material layer, the amount of the binder may be about 1 wt % to about 5 wt % based on a total weight of the negative electrode active material layer. When it further includes the conductive material, it may include about 90 wt % to about 98 wt % of the negative electrode active material, about 1 wt % to about 5 wt % of the binder, and about 1 wt % to about 5 wt % of the conductive material.

The binder improves binding properties of negative electrode active material particles with one another and with a negative electrode current collector. The binder may be a non-water-soluble binder, a water-soluble binder, or a combination thereof.

The non-water-soluble binder may be polyvinylchloride, carboxylated polyvinylchloride, polyvinylfluoride, polyurethane, polytetrafluoroethylene, polyvinylidene fluoride, polyethylene, polypropylene, polyamideimide, polyimide, or a combination thereof.

The water-soluble binder may be a rubber-based binder and/or a polymer resin binder. The rubber-based binder may be selected from a styrene-butadiene rubber, an acrylated styrene-butadiene rubber (SBR), an acrylonitrile-butadiene rubber, an acrylic rubber, a butyl rubber, a fluorine rubber, and a combination thereof. The polymer resin binder may be selected from polytetrafluoroethylene, an ethylenepropylene copolymer, polyethyleneoxide, polyvinylpyrrolidone, polyepichlorohydrine, polyphosphazene, polyacrylonitrile, polystyrene, ethylenepropylenedienecopolymer, polyvinylpyridine, chlorosulfonatedpolyethylene, latex, a polyester resin, an acrylic resin, a phenolic resin, an epoxy resin, polyvinyl alcohol, and a combination thereof.

When the water-soluble binder is used as the negative electrode binder, a cellulose-based compound may be further used to provide viscosity as a thickener. The cellulose-based compound includes one or more selected from carboxylmethyl cellulose, hydroxypropylmethyl cellulose, methyl cellulose, and an alkali metal salt thereof. The alkali metal may be Na, K, and/or Li. Such a thickener may be included in an amount of about 0.1 parts by weight to about 3 parts by weight based on 100 parts by weight of the negative electrode active material.

The conductive material is used to impart conductivity (e.g., electrical conductivity) to the electrode, and in the configured battery, any suitable material that does not cause chemical change (e.g., substantially does not cause an undesirable chemical change in the rechargeable lithium battery) and is electrically conductive may be used. For example, the conductive material may be a carbon-based material such as natural graphite, artificial graphite, carbon black, acetylene black, ketjen black, and/or a carbon fiber; a metal-based material such as a metal powder and/or a metal fiber including copper, nickel, aluminum, and/or silver; a conductive polymer such as a polyphenylene derivative; and/or a mixture thereof.

The negative electrode current collector may be selected from a copper foil, a nickel foil, a stainless steel foil, a titanium foil, a nickel foam, a copper foam, a polymer substrate coated with a conductive metal (e.g., an electrically conductive material), and a combination thereof.

Depending on the type or kind of rechargeable lithium battery, a separator may be present between the positive electrode and the negative electrode. Such a separator material may include polyethylene, polypropylene, polyvinylidene fluoride, and/or multi-layers thereof such as a polyethylene/polypropylene double-layered separator, a polyethylene/polypropylene/polyethylene triple-layered separator, or a polypropylene/polyethylene/polypropylene triple-layered separator.

The rechargeable lithium battery may have a charging upper limit voltage of greater than or equal to about 4.35 V. For example, the charging upper limit voltage may be about 4.35 V to about 4.55 V.

Hereinafter, examples of the present disclosure and comparative examples are described. These examples, however, are not in any sense to be interpreted as limiting the scope of the disclosure.

### Manufacture of Rechargeable Lithium Battery Cells

### Example 1

LiNi_{0.75}Mn_{0.23}Al_{0.02}O₂ as a positive electrode active material, polyvinylidene fluoride as a binder, and acetylene black as a conductive material were mixed together in a weight ratio of 96:3:1 and then, dispersed in N-methyl pyrrolidone, thereby preparing a positive electrode active material slurry.

The positive electrode active material slurry was coated on a 15 μm-thick Al foil, dried at 100° C., and pressed, thereby manufacturing a positive electrode.

A negative electrode active material slurry was prepared by using a mixture of artificial graphite and an Si—C composite in a weight ratio of 93:7 as a negative electrode active material, mixing together the negative electrode active material, a styrene-butadiene rubber binder, and carboxylmethyl cellulose in a weight ratio of 98:1:1, and dispersing the obtained mixture in distilled water.

The Si composite had a core including artificial graphite and silicon particles and coated with coal pitch on the surface.

The negative electrode active material slurry was coated on a 10 μm-thick Cu foil, dried at 100° C., and pressed, thereby manufacturing a negative electrode.

The manufactured positive and negative electrodes were assembled with a 10 μm-thick polyethylene separator to manufacture an electrode assembly, and an electrolyte solution was injected thereinto, thereby manufacturing a rechargeable lithium battery cell.

The composition of the electrolyte solution was as follows.

### Composition of Electrolyte Solution

Lithium salt: 1.5 M LiPF₆
Non-aqueous organic solvent: ethylmethyl carbonate:dimethyl carbonate (EMC:DMC=weight ratio of 20:80)
Additive: 0.25 parts by weight of compound represented by Chemical Formula 1-1/10 parts by weight of fluoroethylene carbonate (FEC)/0.5 parts by weight of succinonitrile (SN)

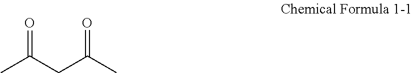

(in the composition of the electrolyte solution, “parts by weight” means a relative weight of an additive based on 100 weights of a total weight of an electrolyte solution excluding the additive (lithium salt+non-aqueous organic solvent).)

### Example 2

A rechargeable lithium battery cell was manufactured in the same manner as Example 1 except that the content of the compound represented by Chemical Formula 1-1 in the composition of the electrolyte solution was changed into 0.5 parts by weight.

### Example 3

A rechargeable lithium battery cell was manufactured in the same manner as Example 1 except that dimethyl carbonate (DMC) alone was used as the non-aqueous organic solvent in the composition of the electrolyte solution.

### Example 4

A rechargeable lithium battery cell was manufactured in the same manner as Example 1 except that the content of the compound represented by Chemical Formula 1-1 in the composition of the electrolyte solution was changed into 0.5 parts by weight, and the dimethyl carbonate (DMC) alone was used as the non-aqueous organic solvent.

### Example 5

A rechargeable lithium battery cell was manufactured in the same manner as Example 1 except that 0.5 parts by weight of the compound represented by Chemical Formula 1-2 was used instead of the compound represented by Chemical Formula 1-1 in the composition of the electrolyte solution.

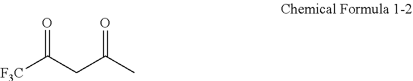

### Example 6

A rechargeable lithium battery cell was manufactured in the same manner as Example 1 except that 0.5 parts by weight of the compound represented by Chemical Formula 1-3 was used instead of the compound represented by Chemical Formula 1-1 in the composition of the electrolyte solution.

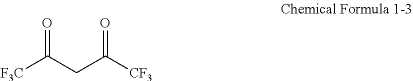 [SMILES: O=C(CC(=O)C(F)(F)F)C(F)(F)F]

### Comparative Example 1

A rechargeable lithium battery cell was manufactured in the same manner as Example 1 except that the compound represented by Chemical Formula 1-1 was not used in the composition of the electrolyte solution.

### Comparative Example 2

A rechargeable lithium battery cell was manufactured in the same manner as Example 1 except that ethylene carbonate:ethylmethyl carbonate:dimethyl carbonate (in a weight ratio of EC:EMC:DMC=20:10:70) were used as the non-aqueous organic solvent in the composition of the electrolyte solution.

### Evaluation 1: Evaluation of Room-Temperature Charge/Discharge Cycle Characteristics

The rechargeable lithium battery cells according to Examples 1 to 6 and Comparative Examples 1 to 2 were charged and discharged under the following conditions and evaluated with respect to cycle characteristics, and the results are shown in Table 1.

After performing 200 cycles of the charge and discharge under the 0.33 C charge (CC/CV, 4.45 V, 0.025 C cut-off)/1.0 C discharge (CC, 2.5 V cut-off) conditions at 25° C., the cells were measured with respect to changes in capacity retention rate and direct current internal resistance (DC-IR) change.

DC-IR was calculated according to Equations 1 and 2 based on a voltage changed by discharging the cells, while applying a current of SOC 50% C for 30 seconds, and the results are shown in Table 1.

Capacity retention rate=(capacity after 200 cycles/capacity after 1 cycle)*100 Equation 1

DC-IR change=(DC-IR after 200 cycles)/(DC-IR after 1 cycle)*100 Equation 2

TABLE 1

| | Capacity | DC-IR | DC-IR | |
|---|---|---|---|---|
| | retention rate | after 1 | after 200 | DC-IR |
| | (@25° C., | cycle | cycles | change |
| | 200 Cycles) | @25° C. | @25° C. | @25° C. |
| | (%) | (mΩ) | (mΩ) | (%) |
| Example 1 | 91.7 | 43.4 | 84.2 | 194 |
| Example 2 | 92.2 | 43.8 | 83.8 | 191 |
| Example 3 | 92.4 | 45.5 | 85.2 | 187 |
| Example 4 | 93.2 | 46.1 | 84.8 | 184 |
| Example 5 | 95.1 | 44.7 | 81.5 | 182 |
| Example 6 | 93.0 | 45.1 | 82.6 | 183 |
| Comparative | 89.9 | 42.9 | 85.7 | 200 |
| Example 1 | | | | |
| Comparative | 89.8 | 42.8 | 83.4 | 195 |
| Example 2 | | | | |

Referring to Table 1, when the additive according to the present disclosure was used, room-temperature cycle-life characteristics were improved.

### Evaluation 2: Evaluation of High-Temperature (45° C.) Cycle-Life Characteristics

The rechargeable lithium battery cells according to Examples 1 to 6 and Comparative Examples 1 to 2 were 200 cycles charged and discharged under 0.33 C charge (CC/CV, 4.45 V, 0.025 C cut-off)/1.0 C discharge (CC, 2.5 V cut-off) conditions at 45° C. and then, evaluated with respect to in capacity retention rate and direct current internal resistance (DC-IR) change.

DC-IR was calculated according to Equations 1 and 2 based on a voltage changed by discharging the cells, while applying a current of SOC 50% C for 30 seconds, and the results are shown in Table 2.

TABLE 2

| | Capacity | DC-IR | DC-IR | |
|---|---|---|---|---|
| | retention rate | after 1 | after 200 | DC-IR |
| | (@ 45° C., | cycle | cycles | change |
| | 200 Cycles) | @ 45° C. | @ 45° C. | @ 45° C. |
| | (%) | (mΩ) | (mΩ) | (%) |
| Example 1 | 89.6 | 50 | 106 | 212 |
| Example 2 | 90.1 | 50.4 | 105.6 | 210 |
| Example 3 | 90.3 | 52.1 | 107 | 205 |
| Example 4 | 91.1 | 52.7 | 106.6 | 202 |
| Example 5 | 95.1 | 44.7 | 81.5 | 182 |
| Example 6 | 93.0 | 45.1 | 82.6 | 183 |
| Comparative | 87.8 | 49.5 | 107.5 | 217 |
| Example 1 | | | | |
| Comparative | 87.7 | 49.4 | 105.2 | 213 |
| Example 2 | | | | |

Referring to Table 2, when the additive according to the present disclosure was used, high-temperature cycle-life characteristics were improved.

### Evaluation 3: Evaluation of High-Temperature Storage Characteristics (Capacity Retention Rate/Capacity Recovery Rate/DC-IR)

The rechargeable lithium battery cells according to Examples 1 to 6 and Comparative Examples 1 to 2 were once charged and discharged at 0.33 C and then, measured with respect to charge and discharge capacity (before high-temperature storage).

In addition, the rechargeable lithium battery cells according to Examples 1 to 6 and Comparative Examples 1 to 2 were charged to SOC 100% (a charge state to 100% of charge capacity based on 100% of total battery charge capacity) and stored at 60° C. for 30 days, and discharged to 3.0 V at 0.33 C under a constant current condition and then, measured with respect to initial discharge capacity.

The cells were recharged to 4.3 V at 0.33 C under a constant current condition and cut off at 0.02 C under a constant voltage condition and discharged to 3.0 V at 0.33 C under a constant current condition and then, twice measured with respect to discharge capacity. A ratio of the first discharge capacity to the initial discharge capacity was provided as capacity retention rate, and the second discharge capacity was provided as capacity recovery rate.

The rechargeable lithium battery cells according to Examples 1 to 6 and Comparative Examples 1 to 2 were measured with respect to ΔV/ΔI (voltage change/current change) to obtain initial DC internal resistance (DC-IR), fully charged (SOC 100%) to secure an internal maximum energy state, stored at a high temperature of 60° C. for 30 days, and then, measured again with respect to DC internal resistance, which were used to calculate a DC-IR increase rate (%) according to Equation 3, and the results are shown in Table 3.

DCIR increase rate=(DCIR after 30 days/Initial DCIR)*100 Equation 3

TABLE 3

| | Capacity | Capacity | | | |
|---|---|---|---|---|---|
| | retention | recovery | | | |
| | rate | rate | Initial | DC-IR | DC-IR |
| | (@60° C., | (@60° C., | DCIR | @60° C., | increase |
| | 30 D) | 30 D) | (@25° C., | 30 D | rate |
| | (%) | (%) | mΩ) | (mΩ) | (%) |
| Example 1 | 90.8 | 94.2 | 43.4 | 96 | 221 |
| Example 2 | 91.3 | 94.7 | 43.8 | 95.6 | 218 |
| Example 3 | 91.5 | 94.9 | 45.5 | 97 | 213 |
| Example 4 | 92.3 | 95.7 | 46.1 | 96.6 | 210 |
| Example 5 | 96.3 | 97.6 | 44.7 | 93.3 | 209 |
| Example 6 | 94.2 | 95.5 | 45.1 | 94.4 | 209 |
| Comparative | 89 | 92.4 | 42.9 | 97.5 | 227 |
| Example 1 | | | | | |
| Comparative | 88.9 | 92.3 | 42.8 | 95.2 | 222 |
| Example 2 | | | | | |

Referring to Table 3, the rechargeable lithium battery cells according to Examples 1 to 6 exhibited improved capacity retention rate and capacity recovery rate when stored at a high temperature and a resistance variation ratio was suppressed from DC-IR increase rate, when compared to the cells according to Comparative Examples 1 to 2.

While the subject matter of this disclosure has been described in connection with what is presently considered to be practical example embodiments, it is to be understood that the present disclosure is not limited to the disclosed embodiments, but, on the contrary, is intended to cover various modifications and equivalent arrangements included within the spirit and scope of the appended claims, and equivalents thereof.

### DESCRIPTION OF SYMBOLS

100: rechargeable lithium battery
112: negative electrode
113: separator
114: positive electrode
120: battery case
140: sealing member

What is claimed is:

1. A rechargeable lithium battery, comprising:
an electrolyte solution comprising a non-aqueous organic solvent, a lithium salt, and an additive;
a positive electrode comprising a positive electrode active material; and
a negative electrode comprising a negative electrode active material,
wherein the non-aqueous organic solvent comprises ethylene carbonate in an amount of less than about 5 wt %,
the additive comprises a compound represented by Chemical Formula 1, and
the positive electrode active material is represented by Chemical Formula 2: 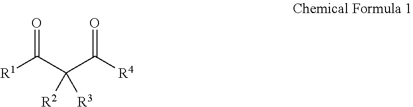
wherein, in Chemical Formula 1,
R¹ to R⁴ are each independently hydrogen, a halogen, a cyano group, a substituted or unsubstituted C1 to C20 alkyl group, a substituted or unsubstituted C6 to C20 aryl group, or a substituted or unsubstituted C2 to C30 heterocyclic group;
LiₐNiₓMn_{1-x-y}A_{y}O_{2±b}X_{c} Chemical Formula 2
wherein, in Chemical Formula 2,
0.9≤a≤1.2, 0≤b≤0.1, 0≤c≤0.1, 0.5≤x≤0.95, and 0≤y<0.3,
A is at least one element selected from B, Na, Mg, Al, Ti, and Si, and
X is at least one element selected from S, F, P, and Cl.

2. The rechargeable lithium battery of claim 1, wherein:
the non-aqueous organic solvent is composed of only chain carbonate.

3. The rechargeable lithium battery of claim 2, wherein:
the chain carbonate is represented by Chemical Formula 3: 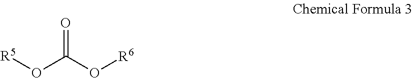
wherein, in Chemical Formula 3
R⁵ and R⁶ are each independently a substituted or unsubstituted C1 to C20 alkyl group.

4. The rechargeable lithium battery of claim 1, wherein:
the non-aqueous organic solvent is a mixed solvent comprising at least two solvents selected from dimethyl carbonate (DMC), diethyl carbonate (DEC), dipropyl carbonate (DPC), methylpropyl carbonate (MPC), ethylpropyl carbonate (EPC), and ethylmethyl carbonate (EMC).

5. The rechargeable lithium battery of claim 1, wherein:
The non-aqueous organic solvent comprises ethylmethyl carbonate (EMC) and dimethyl carbonate (DMC) in a weight ratio of about 0:100 to about 50:50.

6. The rechargeable lithium battery of claim 1, wherein:
at least one selected from R¹ to R⁴ in Chemical Formula 1 is an unsubstituted C1 to C20 alkyl group, a C1 to C20 alkyl group substituted with an electron withdrawing group, an unsubstituted C6 to C20 aryl group, a C6 to C20 aryl group substituted with an electron withdrawing group, an unsubstituted C2 to C30 heterocyclic group, or a C2 to C30 heterocyclic group substituted with an electron withdrawing group.

7. The rechargeable lithium battery of claim 6, wherein:
the electron withdrawing group is at least one selected from a halogen, an isocyanate group (—NCO), an isothiocyanate group (—NCS), a cyanate group (—OCN), a thiocyanate group (—SCN), a cyano group (—CN), an isocyano group (—NC), a —N═C═N— group, a —N═S═N— group, a nitro group (NO₂), a trifluoromethane (CF₃) group, a pentafluoroethane (C₂F₅) group, a trifluoromethane sulfonyl (SO₂CF₃) group, a pentafluoroethane sulfonyl (SO₂C₂F₅) group, a trifluoromethane sulfonate (SO₃CF₃) group, a pentafluoroethane sulfonate (SO₃C₂F₅) group, a pentafluorophenyl (C₆F₅) group, an acetyl (COCH₃) group, an ethyl ketone (COC₂H₅) group, a propyl ketone (COC₃H₇) group, a butyl ketone (COC₄H₉) group, a pentyl ketone (COC₅H₁₁) group, a hexyl ketone (COC₆H₁₃) group, an ethanoate (CO₂CH₃) group, a propanoate (CO₂C₂H₅) group, a butaneoate (CO₂C₃H₇) group, a pentanoate (CO₂C₄H₉) group, and a hexanoate (CO₂C₅H₁₁) group.

8. The rechargeable lithium battery of claim 1, wherein:
at least one selected from R² and R³ in Chemical Formula 1 is hydrogen.

9. The rechargeable lithium battery of claim 1, wherein:
R² and R³ of Chemical Formula 1 are each hydrogen.

10. The rechargeable lithium battery of claim 1, wherein:
the compound represented by Chemical Formula 1 is selected from compounds of Group 1:

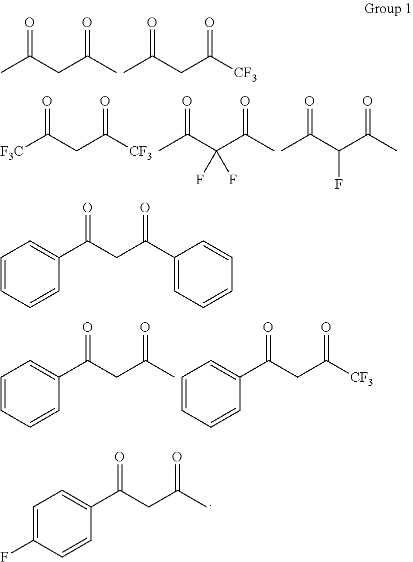

11. The rechargeable lithium battery of claim 1, wherein:
the compound represented by Chemical Formula 1 is included in an amount of about 0.01 to about 5.0 parts by weight based on 100 parts by weight of the electrolyte solution for a rechargeable lithium battery.

12. The rechargeable lithium battery of claim 1, wherein:
the electrolyte solution further comprises at least one other additive selected from vinylene carbonate (VC), fluoroethylene carbonate (FEC), difluoroethylene carbonate, chloroethylene carbonate, dichloroethylene carbonate, bromoethylene carbonate, dibromoethylene carbonate, nitroethylene carbonate, cyanoethylene Carbonate, vinylethylene carbonate (VEC), adiponitrile (AN), succinonitrile (SN), 1,3,6-hexane tricyanide (HTCN), propensultone (PST), propanesultone (PS), lithium tetrafluoroborate (LiBF₄), lithium difluorophosphate (LiPO₂F₂), and 2-fluorobiphenyl (2-FBP).

13. The rechargeable lithium battery of claim 1, wherein:
x in Chemical Formula 2 is 0.8≤x≤0.95.

14. The rechargeable lithium battery of claim 1, wherein:
the positive electrode active material comprises LiNi_{0.75}Mn_{0.25}O₂, LiNi_{0.80}Mn_{0.20}O₂, LiNi_{0.85}Mn_{0.15}O₂, LiNi_{0.90}Mn_{0.10}O₂, or LiNi_{0.95}Mn_{0.05}O₂, or a solid solution thereof.

15. The rechargeable lithium battery of claim 1, wherein:
the negative electrode active material comprises at least one selected from graphite and a Si composite.

16. The rechargeable lithium battery of claim 1, wherein:
the rechargeable lithium battery has a charging upper limit voltage of greater than or equal to about 4.35 V.
